(19) United States
(12) Patent Application Publication
(10) Pub. No.: US 2018/0333860 A1
(43) Pub. Date: Nov. 22, 2018

(54) SYSTEM AND METHOD FOR PRESCRIPTION MEDICINE DELIVERY

(71) Applicant: TRANSLOGIC CORPORATION, Broomfield, CO (US)

(72) Inventors: BOBBI JAMRISKA, Broomfield, CO (US); ALEXANDRIA HETTLER, Glendale, CO (US); STEPHAN SONDEREGGER, Denver, CO (US); RAYMOND ANTHONY CASTRO, Parker, CO (US)

(21) Appl. No.: 15/983,149

(22) Filed: May 18, 2018

Related U.S. Application Data
(60) Provisional application No. 62/508,052, filed on May 18, 2017.

(51) Int. Cl. B25J 11/00; G07F 17/00; G16H 20/10

(52) U.S. Cl. CPC B25J 11/009; G16H 20/10; G07F 17/0092

(57) ABSTRACT

Systems and methods are presented that problems with medication nonadherence by securely providing prescription medications directly to a discharging patient while also providing a session with a prescription consultant for any required or requested consultation. In an arrangement, an automated mobile robot (AMR) securely transports prescription drugs or medications from the pharmacy to a patient discharge location. In one arrangement, a discharging patient utilizes a telemedicine interface supported by the AMR to have a remote consultation session with a pharmacist. Once the remote consultation is performed, the patient may be provided access to prescription medicines secured within the AMR.

### CROSS REFERENCE

The present application claims the benefit of the filing date of U.S. Provisional Patent No. 62/508,052 having a filing date of May 18, 2015, the entire contents of which is incorporated herein by reference.

### FIELD

The present disclosure relates to the delivery of pharmaceuticals. More specifically the disclosure relates to a system and method for the automated transport of prescription medications or other substances to patients while being discharged from a care facility. Once transported to the patient, the patient is provided access to the prescription medications upon completion of a consultation with a pharmacist or other medical professional.

### BACKGROUND

Nonadherence with medication regimens often results in increased use of medical resources, such as physician visits, laboratory tests, unnecessary additional treatments, emergency department visits, and/or hospital/nursing home (e.g., care facility) admissions or re-admissions. Nonadherence may also result in treatment failure. According to some sources, medication nonadherence costs over four billion dollars annually in the United States alone. Further, 35% of all acute care readmissions within 30 days of discharge occur due to medication nonadherence. Likewise, 11% of all hospital readmissions within 30 days occur due to medication nonadherence. Patients may be nonadherent during different stages of their treatment. However, one primary cause of nonadherence is the failure of many patients to fill their prescriptions. More specifically, 23% of patients discharged from a care facility fail to ever fill their prescription(s).

### SUMMARY

The present disclosure is broadly directed to delivering prescription medications to patients in conjunction with their discharge from a care facility. The provision of prescription medications or other substances (hereafter ‘prescription medications’) to patients during discharge will significantly reduce or eliminate the primary cause of medication nonadherence, failure to fill a prescription. Aspects of the systems and methods (i.e., utilities) are based on several recognitions. One recognition is that while many care facilities have an on-site out-patient pharmacy, this pharmacy is not necessarily located where a patient is discharged. Therefore, patients previously had to make an effort to visit the on-site pharmacy after discharge to fill their prescriptions. Rather than taking time to stop at the on-site pharmacy, many patients leave the facility intending to fill their prescriptions at their regular pharmacy. This leads to nonadherence.

It has been further recognize that providing prescription medications to patients during discharge provides additional challenges. For instance, for care facilities having large numbers of patients, manual delivery of prescriptions to each discharging patient would require significant staffing levels to securely courier the prescription medications from the pharmacy to each discharging patient. Additionally, such a courier arrangement has a potential for abuse (e.g., drug diversion). Further, a number of states have pharmacy regulations that require a person filling a prescription (e.g., discharging patient) receive counseling regarding the prescription prior to receiving the prescription medications. Often, such pharmacy regulations require a person filling a prescription receive counseling from a pharmacist or other trained medical professional (e.g., prescription consultant) regarding their prescription medication before dispensing. This consultation provides the prescription consultant the opportunity to educate patients who present new prescriptions and protect them from potential problems associated with a new medication. For instance, the prescription consultant may discuss, among other things, possible side effects, contraindications with other medication and/or the importance of following directions. Stated otherwise, many prescription drugs cannot be delivered to a discharging patient free of consultation with a prescription consultant.

The presented utilities address the problems with medication nonadherence by securely providing prescription medications directly to a discharging patient while also providing a session with a prescription consultant for any required or requested consultation. In one arrangement, a patient utilizes a telemedicine interface (e.g., computer, laptop, tablet or other screen) to have a remote consultation session with a pharmacist. Such a telemedicine interface may allow for two-way communication (e.g., verbal and visual) between the discharging patient and the prescription consultant. In an arrangement, an automated mobile robot (AMR) transports prescription drugs or medications from the pharmacy to the patient discharge location. That is, the AMR is loaded with a discharge prescription for a patient at an on-site pharmacy of the care facility. The AMR is then operative to maneuver to the location of the discharging patient. In one arrangement, the AMR includes a secure receptacle (e.g., locking bin, receptacle, drawer etc.) that holds the prescription(s). In such an arrangement, the prescription drugs are securely maintained by the AMR between the pharmacy and the discharging patient location. Further, access to the prescription drugs within the AMR may be delayed until a prescription consultation is complete. For instance, at the completion of the consultation, the prescription consultant may, in the case of a remote consultation, remotely unlock the AMR to provide the discharging patient access to the prescription medications. Alternatively, the prescription consultant may provide an access code to the discharging patient, who enters the code into an interface on the AMR. In any arrangement, access may be restricted until the consultation is complete. In one particular arrangement, the AMR supports a telemedicine device interface device. In such an arrangement, the AMR and supported telemedicine interface device may be directed to any discharge location. In another arrangement, the patient is directed to a telemedicine interface screen at discharge and an AMR containing the prescription(s) for the discharging patient meets the patient at this location.

The general process of the utility begins when a patient discharge is planned. For instance the process may begin by the issuance of a computerized physician order entry (CPOE) or other clinical user indicating that the patient will be discharged and an approximate time of the discharge. At such time, discharge prescriptions may be prescribed and sent to the outpatient pharmacy of the care facility. The prescriptions may be prepared by the pharmacy. At the appropriate time, an AMR may be loaded with the prescriptions and deployed. As will be appreciated, the AMR may securely hold multiple different prescriptions (e.g., in separate bays, bins etc.) for different discharging patients. In this regard, the AMR need not return to the pharmacy between each discharge. In any arrangement, the AMR travels to the discharge location of a particular patient.

In one arrangement, notifications may be sent indicating medications are available for a discharging patient. Such notifications may be sent to, for example, a delivery manager that directs an AMR to the pharmacy. Further, notifications may be sent to a nurse, doctor or other discharging staff. Once the AMR and the prescription medicines are at the discharge location, the discharging patient may consult a prescription consultant. In an arrangement, the prescription consultant is consulted via the telemedicine system to receive their consultation. In another arrangement, a prescription consultant meets the patient at the discharge location. For instance, the consultant may meet the patient in response to a message sent by the delivery manager. Such a message may include a discharge location and discharge time. At the end of the consultation, access to the drugs within the AMR is provided.

The utilities set forth above may further utilize various computer systems and/or programs (e.g., delivery manager) that coordinates the efforts of prescription writers, pharmacy staff and/or discharging staff in conjunction with control of the AMR and/or a telemedicine interface. In an arrangement, the requests for medications (e.g., prescriptions), the identity of a person writing the prescription, time of prescription filing, the identity of a person filing the prescription, the time the AMR received the prescription medications, the route the AMR takes to a discharge location, the time the AMR releases the prescription medications, the identity of the person receiving the prescription medications and/or the identity of the prescription consultant may be recorded by the delivery manager. In such an arrangement, the delivery may compile a complete chain of custody record for the prescription medication(s). In an arrangement, the delivery manager may receive discharge prescriptions from the prescription writer and forward these to the pharmacy. In a further arrangement, the delivery manager may further be configured to schedule the AMR. For example, the delivery manager may direct an AMR to travel to a pharmacy to pick up one or more discharge prescriptions. Likewise, the delivery manager may provide one or more destination locations to the AMR that correspond with one or more discharging patients. Further, the delivery manager may provide notifications to discharge staff (e.g., medications ready for delivery, medications on route, estimated time of arrival etc.). In an arrangement, the delivery manager may also interface with the telemedicine system. In another arrangement, the delivery manager may interface with a prescription consultant.

### BRIEF DESCRIPTION OF THE DRAWINGS

FIG. 1A illustrates a graphical overview of one embodiment of the presented system and method;

FIG. 2 illustrates one non-limiting embodiment of an automated mobile delivery robot.

FIG. 3 illustrates one process for delivering prescription medications to a discharging patient.

FIG. 4 illustrates a graphical overview of another embodiment of the presented system and method.

### DETAILED DESCRIPTION

Reference will now be made to the accompanying drawings, which at least assist in illustrating the various pertinent features of the presented inventions. The following description is presented for purposes of illustration and description and is not intended to limit the discussed embodiments to the forms disclosed herein. Consequently, variations and modifications commensurate with the following teachings, and skill and knowledge of the relevant art, are within the scope of the presented inventions. The embodiments described herein are further intended to explain the best modes known of practicing the inventions and to enable others skilled in the art to utilize the inventions in such, or other embodiments and with various modifications required by the particular application(s) or use(s) of the presented inventions.

As noted above, medication nonadherence is a significant problem for care facilities. Depending on the type of facility, it may be expected that between about one in ten and about one in three patients will be readmitted within 30 days after discharge due to medication compliance issues. Accordingly, it would be desirable to increase the medication adherence of discharged patients. The most readily available mechanism for increasing medication adherence is ensuring that patients fill their prescriptions at the time of their discharge. That is, ensuring patients are in physical possession of their prescriptions when they leave a care facility. Accordingly, a system and method is provided herein that allows for providing prescription medications to patients at a discharge location while also permitting the patients to receive necessary pharmacist consultations.

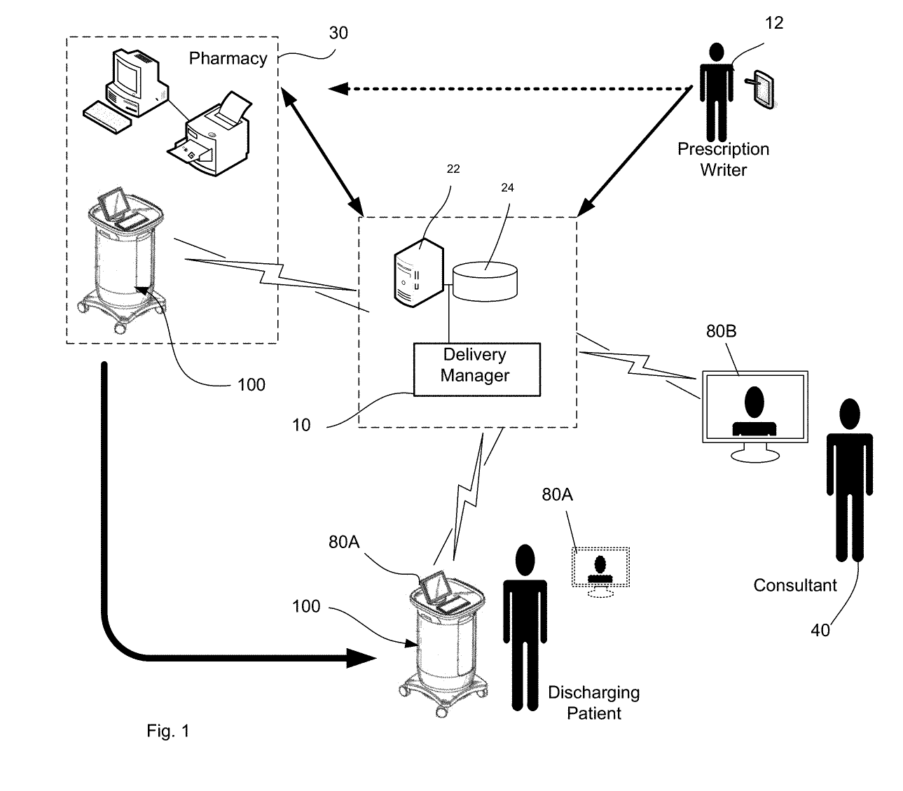

FIG. 1 provide a graphic overview of a system and method utilized to increase medication adherence of patients after discharge by providing these patients their prescription medications at the time of discharge from a care facility. As shown, the operations of several different entities and systems are centrally coordinated by a delivery manager 10. The delivery manager 10 is typically a software platform/program. The delivery manager 10 may be fully automated. However, in other embodiments, the delivery manager may include a user interface and may be at least partially controlled by a user. In the illustrated embodiment, the delivery manager 10 coordinates with a plurality of entities to provide prescription medications to a patient (e.g., discharging patient 50) when that patient is discharging from a care facility. The various entities include a prescription writer 12 (e.g., doctor, physician's assistants, nurse practitioner etc.) who provides one or more prescriptions for a discharging patient, an onsite pharmacy 30 of the care facility that fills the prescriptions for a discharging patient and a consultant 40 (e.g., pharmacist or other trained medical professional), who provides prescription consultation for a discharging patient. In the illustrated embodiment, the consultant 40 interfaces with the discharging patient 50 via a telemedicine system 80A, 80B. In addition, the delivery manager is also in communication with an automated mobile robot (AMR) 100.

The delivery manage 10 can include various hardware and software for implanting the function described herein. The hardware elements can include one or more central processing units (CPUs) 22, input and output (e.g., monitor) devices (not shown). The delivery manager 10 can also include one or more storage devices 24. The device manager 10 can additionally include a computer-readable storage media reader, a communications system such a network card (wireless or wired). The device manager 10 can also include software elements, which may be located within a working memory and include an operating system and/or other code, such as an application program.

The prescription writer 12, pharmacy 30, consultant 40, AMR 100 and telemedicine system 80A, 80B are typically all in data communication with the delivery manager 10. Such data communication may be over any appropriate network including, without limitation, wide area networks, local area networks, wireless networks (e.g., Bluetooth, cellular, etc.), telephonic networks, etc. Further, it will be appreciated that different entities may communicate with the delivery manager utilizing different networks and/or protocols. Collectively, the coordination of these entities and devices allow for providing discharge prescriptions to patients at a discharge locations without increasing staffing levels and while complying with prescription regulations.

The overall process begins when a patient discharge is planned. Initially, during the planning of a discharge, a prescription writer 12 may write a prescription for a discharging patient 50. This prescription may be directed to the delivery manager or the pharmacy 30. In the former regard, the prescription may be provided to the pharmacy 10 by the delivery manager 10. In the latter regard, the pharmacy may contact the delivery manager 10 regarding the impending discharge. Once the prescription is prepared, the pharmacy 30 may notify the delivery manager 10. The delivery manager may then direct an AMR 100 to the pharmacy 30 if an AMR is not already available. Once an AMR 100 is located at the pharmacy and loaded, the delivery manager may provide a location (e.g., room number) and, in an embodiment, a discharge time for the discharging patient. In any embodiment, the AMR 100 may be loaded with the prescriptions and deployed. As will be appreciated, the AMR may securely hold multiple different prescriptions (e.g., in separate bays, bins etc.) for different discharging patients. The AMR 100 navigates to the discharge location of the discharging patient. Notifications may be sent (e.g., to a nurse or other care facility staff) indicating medications are arriving for the discharging patient. Once the AMR 100 and the prescription medicines are at the discharge location, the discharging patient 50 may consult a prescription consultant 40. In the embodiment illustrated in FIG. 1, the prescription consultant 40 is consulted via the telemedicine system 80A, 80B. In this embodiment, the consultation is provided by a remote consultant. At the end of the consultation, access to the drugs within the AMR is provided. For instance, the consultant may authorize the release of the prescription medicines from a secure storage within the AMR 100 and/or provide an access code to the discharging patient that allows the patient to access their prescription medicines within the AMR.

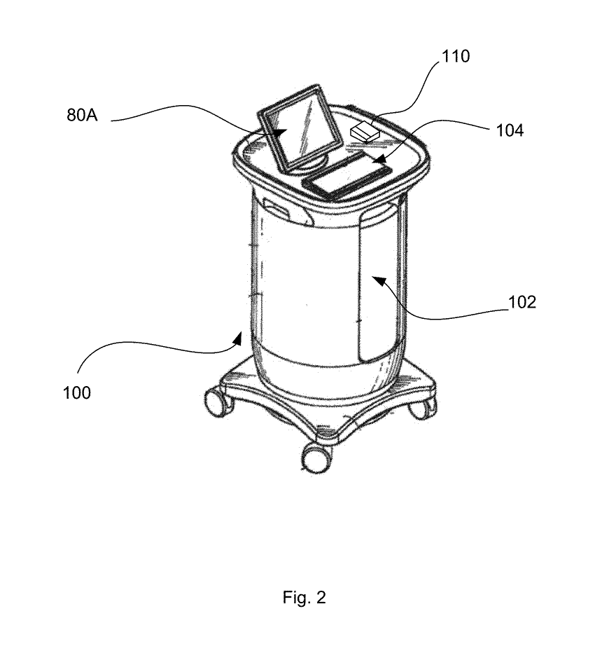

FIG. 2 illustrates one non-limiting embodiment of a delivery AMR 100. The illustrated AMR 100 includes a body with a secure bay or receptacle (e.g., cargo space covered by door 102) formed therein. The AMR 100 includes various sensors for navigating in an environment such as the care facility. For instance, the robot may include various sensors and movement control system attached to and/or disposed within the body. In one arrangement, the movement control system may include a SLAM navigation system. Drive propulsion, battery systems, and at least some portion of the control electronics can be positioned within the body. In any embodiment, the delivery robot can autonomously navigate through changeable indoor or outdoor environments, including but not limited to medical care centers.

In an embodiment, the secure receptacle may be locked to maintain control of custody of items (e.g., prescriptions) placed therein. As shown the secure receptacle 102 includes a locking door formed in a front of the AMR. In addition, the secure receptacle may include multiple individually accessible areas (e.g., drawers, bins, etc.) which may be individually secured and released/opened. The secure receptacle may be accessed when a release authorization input is received by the AMR. In one embodiment, the release authorization input is received from a remote entity (e.g., consultant or delivery manager). In another embodiment, the secure receptacle may be accessed when the release authorization input (e.g., pin code) is entered into a user interface 104 of the AMR. Such a code may, in an embodiment, be provided to the discharging patient during the consultation. In one embodiment, the user interface can be a touchpad, tablet or similar device. In another embodiment, the AMR may include additional interface systems such as scanners (e.g., bar code, RFID, biometric etc.) for use in, for example, confirming the identity of a patient prior to providing access to the secure receptacle. In such an embodiment, the release authorization input may be a confirmation that a scanned patient identification matches the patient identification in the secured receptacle. In another embodiment, the AMR may include a card reader 110. Such a card reader may permit the AMR to receive payments, for example, for the prescription medications. Such payments may be received prior to releasing the medications. One non-limiting example of such a delivery robot is the Relay produced by Savioke of 125 South Market St. Suite 700, San Jose, Calif. 95113, USA. However, it will be appreciated that the present disclosure is not limited to any particular delivery robot/AMR.

The telemedicine system 80A, 80B allows a remote consultant to communicate with a discharging patient. Both the discharging patient and the consultant have access to a screen 80A, 80B, which allow two-way visual and verbal communication. See FIG. 1. In the illustrated embodiment, the discharging patient screen 80A may be supported by the AMR or may be a stand-alone screen (e.g., monitor) and/or a mobile screen (e.g., tablet provided by discharging staff). In one arrangement, the telemedicine system interfaces through the delivery manager to connect a discharging patient with an available consultant once the AMR has arrived at the patient discharge location. Other embodiments may provide direct communications bypassing the delivery manager. One non-limiting telemedicine pharmacist system is PipelineRx, which is located at 600 California Street, Suite 520, San Francisco, Calif. 94108.

The delivery manager 10 effectively act as traffic management for the discharge process. Further, the delivery manager may records all interactions. That is, the request for prescription medications, the identity of a prescription writer, availability of a filled prescription, identity of the person filling the prescription, time the prescription was loaded on the AMR, the route the AMR travels, the discharge location, the identity of the discharging patient, time of discharge and/or the identity of the consultant may be recorded by the delivery manager 10. Stated otherwise, the delivery manager 10 may track the movement of the discharge prescriptions through a facility. Along these lines, system user may have ready access to identify the status of discharge medications (e.g., awaiting preparation, prepared, loaded for delivery, on route, etc.). The delivery manager may provide complete chain of custody information for a discharge prescription identifying, for example, the time and location where a prescription was loaded into the AMR, the identity of the person who loaded the AMR, the route the AMR takes to a discharging patient, the arrival time, the time and/or identity of a person accessing the AMR to remove the prescription. The system may incorporate RDIF or barcode technology to confirm insertion/removal of items into/from the AMR. The delivery manager may be HIPPA compliant as well as compliant with impending medication tracking legislation such as the Drug Supply Chain Security Act (DSCSA). One exemplary delivery manager system is the Delivery Manager system of Swisslog Healthcare, having a location at 10825 East 47th Ave, Denver, Colo. 80239.

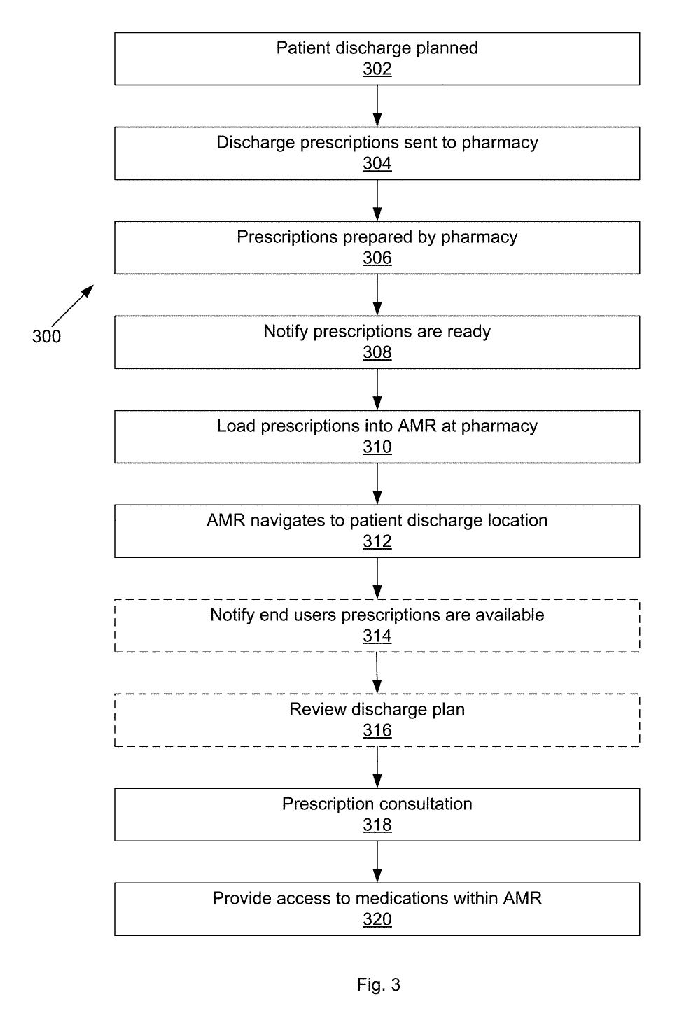

FIG. 3 illustrates one overall process 300 for use in provision of prescription medications to a discharging patient. In one arrangement, upon a patient discharge being planned 302, a prescription writer 12 may prepare and send discharge prescriptions 304 to the delivery manager or alternatively directly to an on-site pharmacy 20. The delivery manager may provide timing information to the pharmacy such that discharge prescriptions are prepared in time for a planned discharge of the patient. In any arrangement, the pharmacy prepares 306 the discharge prescriptions. Once prepared, the delivery manager is informed 308 the prescriptions are available. In conjunction with informing the delivery manager that the prescriptions are available, one or more additional users (e.g., discharge staff) may be informed of an estimated time of arrival to allow, for example, final discharge preparations.

The delivery manager communicates with one or more automated mobile robots (AMR) 100. When a prescription is prepared, the delivery manager may direct an AMR to the pharmacy permitting pharmacy staff to load 310 the discharge prescription into the AMR. At or near the time of discharge, the AMR 100 may travels/navigate 312 to the location of the discharging patient. The delivery location may be provided to the AMR by the delivery manager. Upon arriving or shortly before arriving, an end user (e.g., discharging staff) may be notified 314 that medications are available for discharge. Optionally, discharge plans may be reviewed 316 with the discharging patient. At this time, the discharging patient may contact 318 a consultant, for example, via the telemedicine system. Once the consultation is completed the patient is provided access 320 to the prescription medications within the AMR.

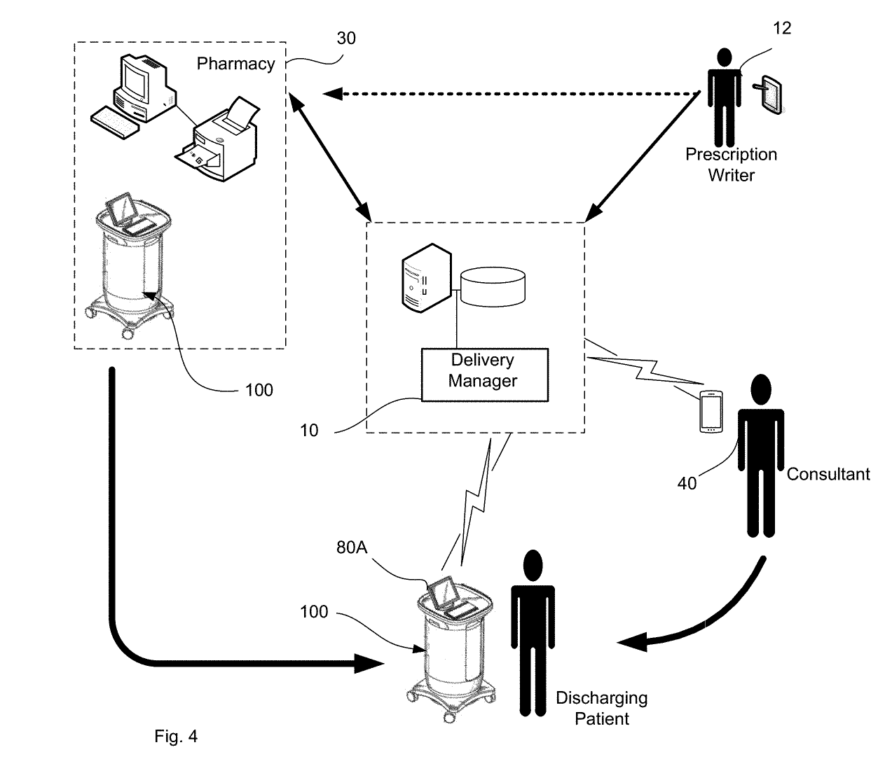

FIG. 4 illustrates an alternate embodiment of a system and method utilized to provide patients their prescription medications at the time of discharge form a care facility. As shown, the system again utilizes a delivery manager to coordinate different entities, such as the pharmacy 20, prescription writer 12 and consultant 40, with an AMR 100 to effect delivery of prescription medications to a discharging patient. However, in this embodiment, rather than utilizing the telemedicine system for remote consultation, the delivery manager communicates with a mobile device 42 of the consultant. In such an embodiment, the delivery manager sends a message the consultant regarding the location of the discharging patient and the discharge time for the patient. In such an arrangement, the consultant may meet the patient in person during the discharge to provide the consultation.

Though described particularly in relation the delivery manager and AMR to deliver prescription medications to a discharging patient, it will be appreciated that the delivery manager and AMR may be utilized for other functions. For instance the delivery manager and AMR may be utilized for ad-hoc secure deliveries of patient specific medications from an inpatient pharmacy to patient care areas for those items that cannot be readily sent by other means. For instance, the system may be utilized when items are too large for a pneumatic transport system, there is no other automated transport system for a delivery location, there are concerns about agitation during pneumatic transport and/or there items are high value or otherwise restricted. In these instances, it may be important for sender to know that the payload has been delivered and who has taken possession of that physical payload. That is, chain of custody may be desired and may be provided by the delivery manager and AMR.

Another application for the delivery manager and AMR is bulk delivery of medications for scheduled replenishment of on-ward medication cabinets to from inpatient pharmacy to patient care areas. In these cases it is also important for sender to know that the payload has been delivered and who has taken possession of that physical payload (chain of custody) as the delivery destination will typically be in an access controlled med room. Another application for the delivery manager and AMR is ad-hoc secure delivery of patient specific blood products from blood bank to patient care areas.

The foregoing description has been presented for purposes of illustration and description. Furthermore, the description is not intended to limit the inventions and/or aspects of the inventions to the forms disclosed herein. Consequently, variations and modifications commensurate with the above teachings, and skill and knowledge of the relevant art, are within the scope of the presented inventions. The embodiments described hereinabove are further intended to explain best modes known of practicing the inventions and to enable others skilled in the art to utilize the inventions in such, or other embodiments and with various modifications required by the particular application(s) or use(s) of the presented inventions. It is intended that the appended claims be construed to include alternative embodiments to the extent permitted by the prior art.

What is claimed is:

1. A method for delivery of prescription medications in a care facility, comprising:
directing an Automated Mobile Robot (AMR) to a location where a discharge prescription has been prepared for a discharging patient, wherein prescription medications associated with the discharge prescription are subsequently loaded into a secure receptacle of the AMR;
deploying the AMR from the location where the prescription medications are loaded into the secure receptacle of the AMR, wherein the AMR travels to a discharge location of the discharging patient;
releasing the prescription medications from the secure receptacle of the AMR after a consultation between the discharging patient and a prescription consultant at the discharge location.

2. The method of claim 1, further comprising:
establishing a communications link between the discharging patient and the prescription consultant, wherein the consultation is a remote consultation provided via the communications link.

3. The method of claim 1, further comprising:
sending a message to the consultant via a network, wherein the message identifies the discharge location and a discharge time.

4. The method of claim 1, wherein releasing the prescription medications comprises:
receiving a release authorization input at the AMR; and
providing access to the secure receptacle of the AMR containing the prescription medications.

5. The method of claim 4, wherein receiving the release authorization input comprises:
receiving an access code entered into a user interface of the AMR.

6. The method of claim 4, wherein receiving the release authorization input comprises:
receiving the release authorization input via a wireless communications link.

7. The method of claim 4, wherein receiving the release authorization input comprises:
confirming a scanned patient identifier matches a patient identifier for the prescription medication within the secure receptacle.

8. The method of claim 1, further comprising, prior to directing the AMR to the location where the discharge prescription has been prepared for the discharging patient:
receiving an indication that a discharge prescription for the discharging patient is ready for delivery.

9. The method of claim 1, further comprising, prior to releasing the prescription medications from the secure receptacle of the AMR:
accepting payment for the prescription medications via an input of the AMR.

10. The method of claim 1, further comprising:
deploying a user interface of a telemedicine system on the AMR, wherein the discharging patient accesses the telemedicine system via the user interface on the AMR for the consultation.

11. The method of claim 10, further comprising:
providing audio and video communications via the user interface.

12. The method of claim 1, further comprising:
receiving and storing information regarding each interaction with the AMR to generate a chain of custody record.

13. A system for delivery of prescription medications in a care facility, comprising:
an Automated Mobile Robot (AMR) configured to navigate within a patient care facility, the AMR including:
a secure receptacle for receiving a payload; and
a display screen; and
an input device
a delivery manager configured to wirelessly communicate with the AMR, wherein the delivery manager is configured to:
receive an input indicating prescription medication are available for a discharging patient;
send wireless communications to the AMR to direct the AMR to a location where the prescription medications are available, wherein the AMR navigates to the location;
send wireless communications to the AMR identifying a discharge location of the discharging patent, wherein the AMR navigates to the discharge location;
wherein, after navigating to the discharge location, the AMR releases the prescription medications to the discharging patient in response to release authorization input.

14. The system of claim 13, wherein the AMR further comprises:
comprises a two-way audio and video system interface, wherein the two-way audio and video system is part of a telemedicine system.
